(19) United States
(12) Patent Application Publication
(10) Pub. No.: US 2022/0059223 A1
(43) Pub. Date: Feb. 24, 2022

(54) EVOLVING SYMPTOM-DISEASE PREDICTION SYSTEM FOR SMART HEALTHCARE DECISION SUPPORT SYSTEM

(71) Applicant: UNIVERSITY-INDUSTRY COOPERATION GROUP OF KYUNG HEE UNIVERSITY, Yongin-si (KR)

(72) Inventors: Choong Seon HONG, Yongin-si (KR); Thar KYI, Yongin-si (KR); Yumin PARK, Suwon-si (KR)

(73) Assignee: UNIVERSITY-INDUSTRY COOPERATION GROUP OF KYUNG HEE UNIVERSITY, Yongin-si (KR)

(21) Appl. No.: 17/002,485

(22) Filed: Aug. 25, 2020

(30) Foreign Application Priority Data
Aug. 24, 2020 (KR) 10-2020-0105957

(51) Int. Cl. G16H 50/20; G16H 10/20; G16H 70/60; G16H 50/70; G16H 10/60; G16H 40/67

(52) U.S. Cl. CPC G16H 50/20; G16H 10/20; G16H 40/67; G16H 50/70; G16H 10/60; G16H 70/60

(57) ABSTRACT

Provided is an evolving symptom-disease prediction system for a smart healthcare decision support system according to the present disclosure. The symptom-disease prediction system may include a client configured to transmit data related to symptom information; and a server configured to detect and predict a disease based on the data related to the symptom information. The server may include a processor configured to, when a disease predicted based on a machine learning model is determined as an existing predicted disease and a new disease, update the machine learning model by aggregating the machine learning model with other models through a model aggregation process shared with other medical institutions.

### CROSS-REFERENCE TO RELATED APPLICATION

This application claims priority from Korean Patent Application No. 10-2020-0105957 filed on Aug. 24, 2020 in the Korean Intellectual Property Office, and all the benefits accruing therefrom under 35 U.S.C. 119, the contents of which in its entirety are herein incorporated by reference.

### TECHNICAL FIELD

At least one example embodiment relates to a symptom-disease prediction system, and more particularly, to an evolving symptom-disease prediction system for a smart healthcare decision support system.

### RELATED ART

Before directly visiting a medical institution to verify diagnosis of a disease, it is necessary to predict problems of one's own disease and physical conditions through the Internet. However, it is difficult to accurately diagnose a disease by simply conducting a search at a search site.

Meanwhile, even in the case of performing symptom-disease prediction using a user terminal, it is difficult to accurately predict a disease from acquired symptoms through the user terminal. In particular, there is no clear method for a comprehensive symptom-disease system that may perform accurate detection and prediction according to a recent epidemic disease and may also perform diagnosis/prevention.

On the other hand, with regard to symptom-disease prediction, efforts are being made to diagnose a condition of a patient based on artificial intelligence (AI) techniques, such as machine learning, as well as statistical methods, such as disease risk scoring. Here, a user may easily diagnose a health status of the user and predict a disease using a user terminal of the user based on techniques, such as AI. However, this symptom-disease prediction method may not apply the recent diagnosis and prediction techniques.

### DETAILED DESCRIPTION

### Technical Subject

Therefore, to outperform the aforementioned issues, an aspect of the present disclosure is to provide an evolving symptom-disease prediction system for a smart healthcare decision support system.

Also, an aspect of the present disclosure is to have an evolving symptom-disease prediction model that maximizes prediction accuracy and is not required to be retrained at a zero level although a new input feature and a target label are introduced.

Also, an aspect of the present disclosure is to have a privacy-aware evolving symptom-disease prediction model that uses a minimum amount of data of a patient.

### Technical Solution

Provided is an evolving symptom-disease prediction system for a smart healthcare decision support system according to the present disclosure. The symptom-disease prediction system may include a client configured to transmit data related to symptom information; and a server configured to detect and predict a disease based on the data related to the symptom information. The server may include a processor configured to, when a disease predicted based on a machine learning model is determined as an existing predicted disease and a new disease, update the machine learning model by aggregating the machine learning model with other models through a model aggregation process shared with other medical institutions.

According to an example embodiment, the symptom-disease prediction system may further include a data storage configured to store medical device data related to a medical device and user information including symptom information; and a model storage configured to store a machine learning model for disease prediction through interaction with a training and calibration pipeline associated with training and calibration for a user data set and a utilization pipeline associated with the disease prediction.

According to an example embodiment, the server may be configured to update the machine learning model stored in the data storage based on local collection data related to the new disease, finally update the machine learning model stored in the data storage based on collection data related to the new disease received from a plurality of medical institutions, and perform detection and prediction of the new disease through the finally updated machine learning model and distribute updated model information to a plurality of medical institution servers corresponding to the plurality of medical institutions.

According to an example embodiment, the server may be configured to update the machine learning model stored in the data storage based on local collection data related to the new disease, finally update the machine learning model stored in the data storage based on collection data related to the new disease received from a central system that is a representative medical institution among the plurality of medical institutions, and perform detection and prediction of the new disease through the finally updated machine learning model and distribute updated model information to a server of the representative medical institution.

According to an example embodiment, the server may be configured to update the machine learning model stored in the data storage based on local collection data related to the new disease, finally update the machine learning model stored in the data storage based on collection data related to the new disease received from a subsystem that is a representative medical institution of a group to which the server belongs, and perform detection and prediction of the new disease through the finally updated machine learning model and distribute updated model information to a server of the subsystem that is the representative medical institution of the group.

According to an example embodiment, the server may be configured to when update of the machine learning model is evaluated to not be performed based on the collection data related to the new disease received from the subsystem, receive second collection data from the central system interacting with subsystems of each group through the subsystem, re-update the machine learning model stored in the data storage based on the second collection data, and perform detection and prediction of the new disease through the re-updated machine learning model and distribute re-updated model information to the server of the subsystem that is the representative medical institution of the group.

According to an example embodiment, the server may be configured to detect a first point in time at which the predicted disease is determined as the existing predicted disease and the new disease, and control the machine learning model to be trained based on data acquired after the first point in time, at a second point in time after the first point in time.

According to an example embodiment, the server may be configured to detect a first point in time at which the predicted disease is determined as the existing predicted disease and the new disease, control the machine learning model to be trained based on user data of a corresponding medical institution when performing training and calibration of the user data set, at a second point in time after the first point in time, and control the machine learning model to be trained based on disease data of a corresponding medical institution and other medical institutions acquired after the first point in time, at the second point in time.

According to an example embodiment, the server may be configured to perform prediction and detection of the new disease using the updated machine learning model, and transmit, to the client that is a user terminal associated with a user of which the new disease is detected, detection results about the new disease and diagnostic results and prevention information according to body and health information of the user.

Provided is a server of an evolving symptom-disease prediction system for a smart healthcare decision support system according to another aspect of the present disclosure. The server may include a storage configured to store medical device data related to a medical device and user information including symptom information, and to store a machine learning model for disease prediction through interaction with a training and calibration pipeline associated with training and calibration for a user data set and a utilization pipeline associated with the disease prediction; and a processor configured to, when a disease predicted based on the machine learning model is determined as an existing predicted disease and a new disease, control the machine learning model to be updated by aggregating the machine learning model with other models through a model aggregation process shared with other medical institutions.

According to an example embodiment, the storage may include a data storage configured to store the medical device data related to the medical device and the user information including the symptom information; and a model storage configured to store the machine learning model for the disease prediction through interaction with the training and calibration pipeline associated with training and calibration for the user data set and the utilization pipeline associated with the disease prediction.

According to an example embodiment, the processor may be configured to update the machine learning model stored in the data storage based on local collection data related to the new disease, finally update the machine learning model stored in the data storage based on collection data related to the new disease received from a plurality of medical institutions, and perform detection and prediction of the new disease through the finally updated machine learning model and distribute the updated model information to a plurality of medical institution servers corresponding to the plurality of medical institutions.

According to an example embodiment, the processor may be configured to update the machine learning model stored in the data storage based on local collection data related to the new disease, finally update the machine learning model stored in the data storage based on collection data related to the new disease received from a central system that is a representative medical institution among the plurality of medical institutions, and perform detection and prediction of the new disease through the finally updated machine learning model and distribute updated model information to a server of the representative medical institution.

According to an example embodiment, the processor may be configured to update the machine learning model stored in the data storage based on local collection data related to the new disease, finally update the machine learning model stored in the data storage based on collection data related to the new disease received from a subsystem that is a representative medical institution of a group to which the server belongs, and perform detection and prediction of the new disease through the finally updated machine learning model and distribute updated model information to a server of the subsystem that is the representative medical institution of the group.

According to an example embodiment, the processor may be configured to, when update of the machine learning model is evaluated to not be performed based on the collection data related to the new disease received from the subsystem, receive second collection data from the central system interacting with subsystems of each group through the subsystem, re-update the machine learning model stored in the data storage based on the second collection data, and perform detection and prediction of the new disease through the re-updated machine learning model and distribute re-updated model information to the server of the subsystem that is the representative medical institution of the group.

According to an example embodiment, the processor may be configured to detect a first point in time at which the predicted disease is determined as the existing predicted disease and the new disease, and control the machine learning model to be trained based on data acquired after the first point in time, at a second point in time after the first point in time.

According to an example embodiment, the processor may be configured to detect a first point in time at which the predicted disease is determined as the existing predicted disease and the new disease, control the machine learning model to be trained based on user data of a corresponding medical institution when performing training and calibration of the user data set, at a second point in time after the first point in time, and control the machine learning model to be trained based on disease data of a corresponding medical institution and other medical institutions acquired after the first point in time, at the second point in time.

According to an example embodiment, the processor may be configured to perform prediction and detection of the new disease using the updated machine learning model, and transmit, to a client that is a user terminal associated with a user of which the new disease is detected, detection results about the new disease and diagnostic results and prevention information according to body and health information of the user.

Provided is an evolving symptom-disease prediction method for a smart healthcare decision support system according to still another aspect of the present disclosure. The method may include a user information generation process of generating medical device data related to a medical device and user information including symptom information; a machine learning model generation process of generating a machine learning model for disease prediction through interaction with a training and calibration pipeline associated with training and calibration for a user data set and a utilization pipeline associated with the disease prediction; a disease decision process of determining whether a disease predicted based on the machine learning model is an existing predicted disease and a new disease; and a machine learning model update process of, when the predicted disease is determined as the existing predicted disease and the new disease, controlling the machine learning model to be updated by aggregating the machine learning model with other models through a model aggregation process shared with other medical institutions.

According to an example embodiment, the machine learning model update process may include a first update process of updating a machine learning model stored in a data storage based on local collection data related to the new disease; and a second update process of updating the machine learning model stored in the data storage based on collection data related to the new disease received from a plurality of medical institutions.

According to an example embodiment, the method may further include a disease detection and prediction process of performing detection and prediction of the new disease through a finally updated machine learning model; and a model information distribution process of distributing the finally updated model information to a plurality of medical institution servers corresponding to the plurality of medical institutions.

### Effect

The technical effects of an evolving symptom-disease prediction system for a smart healthcare decision support system according to the present disclosure follow as:

According to the present disclosure, an evolving symptom-disease prediction system for a smart healthcare decision support system may provide various model aggregation methods.

According to the present disclosure, an evolving symptom-disease prediction result for a smart healthcare decision support system may enhance quality of a medical service by providing accurate symptom-disease information to a medical professional.

According to the present disclosure, it is possible to construct a mobile/web application that is a part of a smart healthcare system as a model of a symptom-disease prediction system.

The features and effects of the present disclosure will be apparent through the following detailed description described with reference to the accompanying drawings and thus, those skilled in the art to which the disclosure pertains may easily perform the technical spirit of the disclosure.

### BRIEF DESCRIPTION OF DRAWINGS

FIG. 1 illustrates a system model of a symptom-disease prediction system according to the present disclosure.

FIG. 2 illustrates a configuration of performing training and calibration and utilization of a model through a training and calibration management pipeline and a utilization pipeline of a symptom-disease prediction system according to the present disclosure.

FIGS. 3 to 5 illustrate configurations associated with a model aggregation according to different learning schemes based on different example embodiments.

FIG. 6 is a configuration diagram illustrating interaction between a server of an evolving symptom-disease prediction system for a smart healthcare decision support system and a client according to the present disclosure.

FIG. 7 is a flowchart illustrating an evolving symptom-disease prediction method for a smart healthcare decision support system according to another aspect of the present disclosure.

### MODE

The features and effects of the present disclosure will be apparent through the following detailed description described with reference to the accompanying drawings and thus, those skilled in the art to which the disclosure pertains may easily perform the technical spirit of the disclosure.

The present disclosure may make various alterations and modifications and have some example embodiments and thus, specific example embodiments are illustrated as examples and are described in the detailed description. However, the example embodiments are not construed as being limited to the disclosure and should be understood to include all changes, equivalents, and replacements within the spirit and technical scope of the disclosure.

Regarding reference numerals assigned to elements in the drawings, like reference numerals refer to like elements.

Terms, such as first, second, and the like, may be used herein to describe components. Each of these terminologies is not used to define an essence, order, or sequence of a corresponding component but used merely to distinguish the corresponding component from other component(s).

For example, a first component may be referred to as a second component, and similarly, the second component may also be referred to as the first component without departing from the scope of the disclosure. As used herein, the term “and/or” includes any and all combinations of one or more of the associated listed items.

Unless otherwise defined, all terms, including technical and scientific terms, used herein have the same meaning as commonly understood by one of ordinary skill in the art to which this disclosure pertains.

Terms, such as those defined in commonly used dictionaries, are to be interpreted as having a meaning that is consistent with their meaning in the context of the related art, and are not to be interpreted in an idealized or overly formal sense unless expressly so defined herein.

Also, the suffixes “˜module/block/unit,” etc., used for the following components are assigned or used for ease of preparing the specification only and are not construed to have distinguishing meanings or roles.

Hereinafter, some example embodiments will be described in detail with reference to the accompanying drawings to be easily implemented by those skilled in the art. Also, in the description of example embodiments, detailed description of well-known related structures or functions will be omitted when it is deemed that such description will cause ambiguous interpretation of the present disclosure.

Hereinafter, an evolving symptom-disease prediction system for a smart healthcare decision support system according to the present disclosure is described. Here, one of key elements required to develop a better medical infrastructure is a smart healthcare decision support system capable of providing accurate information to medical professionals. Although various types of map machine learning models may provide accurate information, corresponding models need to be retrained to extend input features (e.g., symptoms, X-ray images, sensor information, and outputs from other models) and target labels (e.g., new diseases). Also, patient data contains sensitive information and thus, cannot be shared with other hospitals to train a model. Accordingly, proposed is a deep learning-based evolving symptom-disease prediction model, that is, a model that protects the privacy of a patient without a need to retrain the model at a zero level when new input features and target labels are introduced.

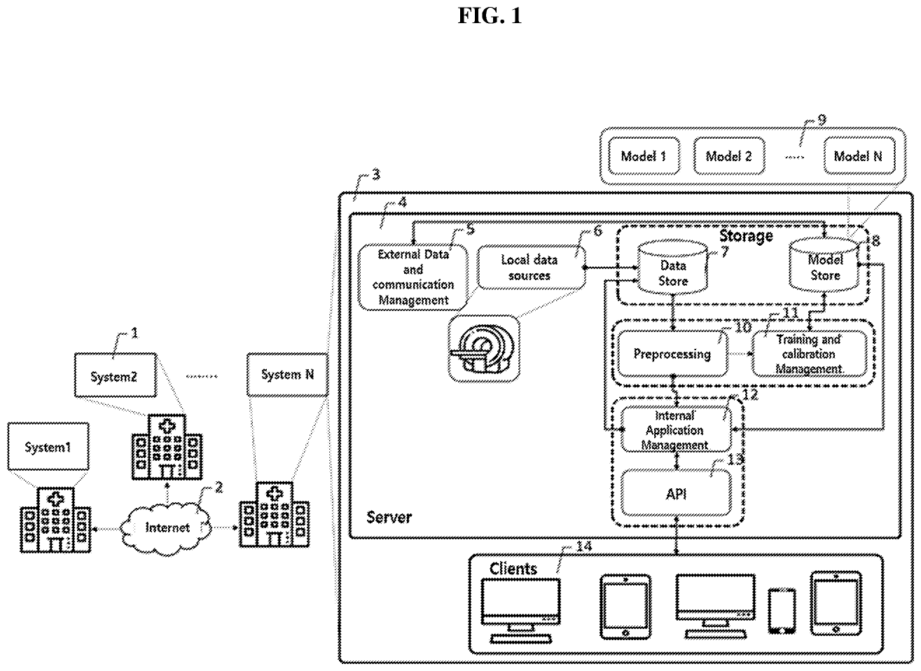

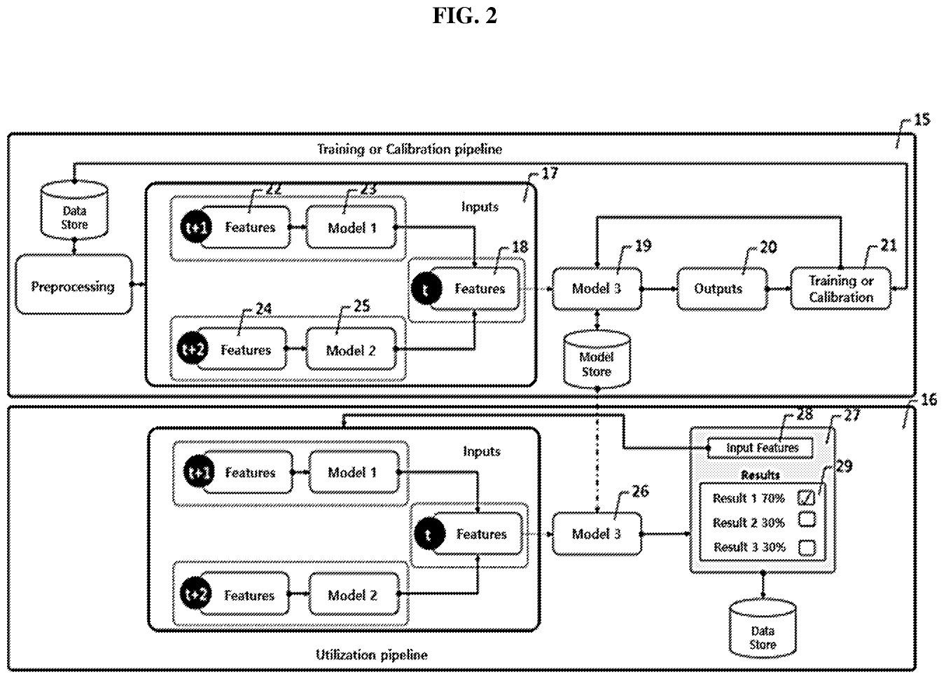

Here, FIG. 1 illustrates a system model of a symptom-disease prediction system according to the present disclosure. Meanwhile, FIG. 2 illustrates a configuration of performing training and calibration and utilization of a model through a training and calibration management pipeline and a utilization pipeline of a symptom-disease prediction system according to the present disclosure.

Since the respective systems that constitute the system model of FIG. 1 are not restricted by locations, hospitals in which the respective systems are present may be in different countries or different regions. The respective systems share model related information instead of sharing patient data through the Internet.

In detail, FIG. 1 illustrates an example in which an evolving symptom-disease prediction system model for a smart healthcare decision support system according to the present disclosure is located in each hospital. The respective hospitals may be located in various regions in the same country (or in different countries) and the respective systems may communicate through the Internet. Each system includes two main portions, that is, a server and a client. The server serves to train, calibrate, and aggregate a machine learning model (e.g., a convolutional neural network (CNN)). Also, the server may provide a learning-based service (e.g., disease classification by symptom) to the client through an application programming interface (API).

Referring to FIG. 1, each system includes the following two major components, that is, a server 3 and a client 14. Referring to FIGS. 1 and 2, each component may be configured as follows.

A plurality of systems 1 may be configured as machine learning-based systems configured to provide different services, such as, for example, an evolving symptom-disease prediction system. Internet 2 may be configured as a communication network configured to communicably connect each system 1. A system 3 configured to predict a corresponding disease among the plurality of systems 1 may correspond to a major component included in the machine learning-based system. Here, the system 3 may be configured as the evolving symptom-disease prediction system.

The server 4 may be configured as a high-end computing machine. The server 4 may include a plurality of components as follows:

An external data and communication management module 5 is configured to manage sharing of machine learning model-related information between geographically distributed systems and model aggregation processes. Local data sources 6 may include various data sources, such as, for example, X-rays, magnetic resonance imaging (MRI), and the like.

A data storage (Data Store) 7 may be configured as a data storage to store data of various sources, such as, for example, X-ray images and MRI images. A model storage (Models Store) 8 may be configured as a model storage to store various machine learning models, such as, for example, a convolutional neural network (CNN) and a recurrent neural network (RNN).

Models 9 may include various machine learning models, such as, for example, a CNN and an RNN. A preprocessing module 10 may be configured as a module to process a variety of data, such as, for example, images, sensor information, and texts.

A training and calibration module 11 may be configured to perform learning and calibration of the machine learning model. An internal application management module 12 may be configured to manage a learning process through utilization pipeline construction for disease prediction. An application programming interface (API) 13 may be configured such that the clients 14 may communicably connect to the server 4. Meanwhile, the clients 14 may include various types of devices, such as, for example, a personal computer (PC), a laptop computer, a tablet, and a smartphone.

A training and calibration pipeline 15 may be configured to train a model using a data set and to calibrate (i.e., correct) the model using data newly collected from the clients. A utilization pipeline 16 may be configured to perform disease prediction and to provide a service, such as disease prediction.

An inputter (Inputs) 17 may be configured to provide features (data) of various sources. Here, a feature portion (Features) 18 may be configured to acquire and provide features at a time t. The feature portion 18 may be configured to acquire and provide input features to train and utilize a model at the time t.

A machine learning model 19 may be configured as a learning model, such as a disease prediction model, configured to classify a disease based on input features. An outputter (Outputs) 20 may be configured to output prediction, classification, or learning results of the machine learning model.

A training and calibration module 21 may be configured to provide features at a time t+1. The features at the time t+1 provided from the training and calibration module 21 are input features newly added to train and utilize a model at the time t+1 to detect a new disease.

Machine learning models 23 and 25 associated with the inputter 17 may be configured as, for example, an X-ray image classification model. Here, feature portions 22 and 24 may be configured to provide features at the time t+1 and features at a time t+2. The features at the time t+2 associated with the feature portion 24 may be a hop number used to transmit a package to reach from a source to a destination.

Meanwhile, a machine learning model according to the present disclosure may be, for example, an MRI image classification model. In this case, the constructed machine learning model may be updated and the updated model may be distributed to the utilization pipeline 16.

With respect to the utilization pipeline 16, a dashboard 27 may be configured as a graphical user interface (GUI) configured to provide information to a client, such as a medical professional. Meanwhile, input information (features) of a client, such as, for example, symptoms and X-ray images, may be provided through a user inputter 28. Results about a disease predicted and detected based on the input may be displayed on an outputter 29.

Meanwhile, the server 3 of the symptom-disease prediction system according to the present disclosure needs to train or calibrate, or aggregate a machine learning model (e.g., a CNN). Also, the server 3 provides a learning-based service (e.g., disease classification based on symptoms) to the client through the application programming interface (API) 13. The server 3 may be a high-end computing device, and the client 14 may be a device, such as, for example, a PC, a laptop computer, a tablet, and a smartphone.

The server 3 includes the external management module 5, the data storage 7, the model storage 8, the preprocessing module 10, the training and calibration management module 11, and the internal application management module 12. A device of the client 14 may access a service by installing software or through an Internet browser. Meanwhile, the preprocessing module 10, the training and calibration management module 11, and the internal application program management module 12 may be inclusively referred to as a processor or a controller. Alternatively, at least one of the preprocessing module 10, the training and calibration management module 11, and the internal application management module 12 may be referred to as a processor or a controller.

FIG. 2 illustrates components of an evolving model training (or) calibration pipeline and an evolving model utilization pipeline. Model utilization may be provided in a server or a client based on a distribution method.

Meanwhile, a system of each medical institution that constitutes the symptom-disease prediction system according to the present disclosure may be installed in a medical institution, such as a hospital. An initial machine learning model may be provided from one of such hospitals. Subsequently, each hospital trains the initial model using data of local patients. For example, at the beginning, by (t) at a time, the server trains the model to learn input symptom information ranging from 0 to 20 and to detect a disease. Next, the trained model is provided to a utilization pipeline and is used to predict a disease based on symptoms and to exhibit results (highly probable disease) with the probability thereof. Users (medical professionals) may select results based on their expertise. A pair of user (medical professional) selected results and input symptom information are stored in a data storage for model calibration.

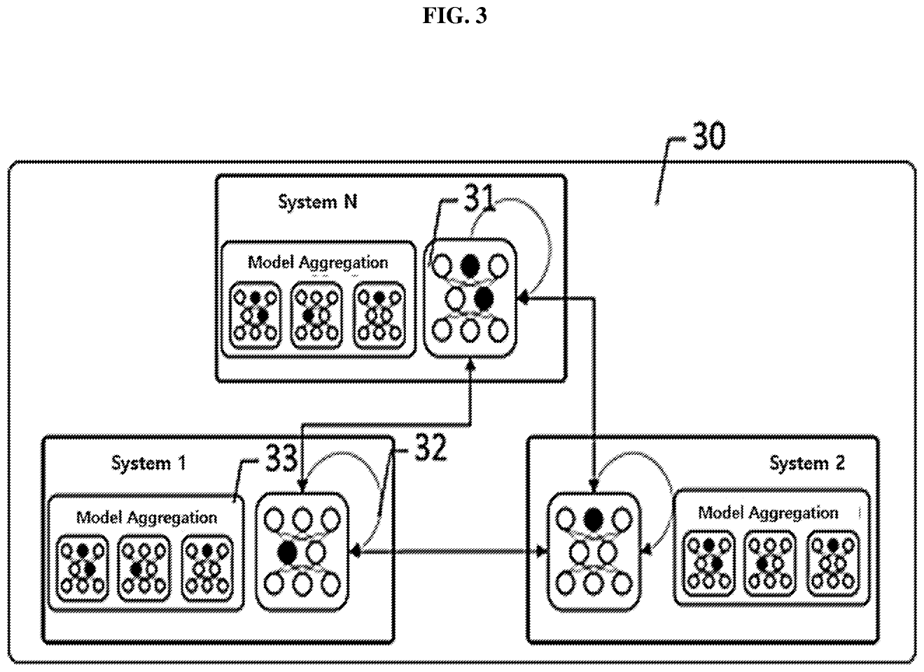

Meanwhile, model aggregation may be performed based on various learning schemes according to the present disclosure. Here, FIGS. 3 to 5 illustrate configurations associated with a model aggregation according to different learning schemes based on different example embodiments.

Referring to FIGS. 3 to 5, updated model parameters are shared with other hospitals and a model of each hospital is enhanced using one of a peer to peer federated learning 30, a classic federated learning 34, and a hierarchical federal federation learning 36.

Here, FIG. 3 illustrates one of aggregation methods available for each system to collect model information from other systems. Next, each system may independently update each model through aggregation with other models. Referring to FIG. 3, the peer to peer federated learning module 30 may perform one of the available aggregation methods to collect model information from other systems. Each system may independently update a model through aggregation with the other models.

The machine learning model 30 may include each machine learning model trained in each hospital. The machine learning model 30 may include, for example, a deep neural network (DNN) and a convolutional neural network (CNN). A local update module 31 may be configured to update (or calibrate) a machine learning model (e.g., a DNN and a CNN) based on local collection data. A model aggregation module 32 may be configured to perform model aggregation based on various arithmetic functions, such as federated averaging.

A basic federated learning module 33 may be configured to perform one of aggregation methods available for a central system to collect model information from other systems. Next, the central system may update a model through aggregation with other models. Next, the central system distributes the updated model information (aggregated model) to all of the other systems. Next, each system independently updates a model through aggregation with an aggregated model.

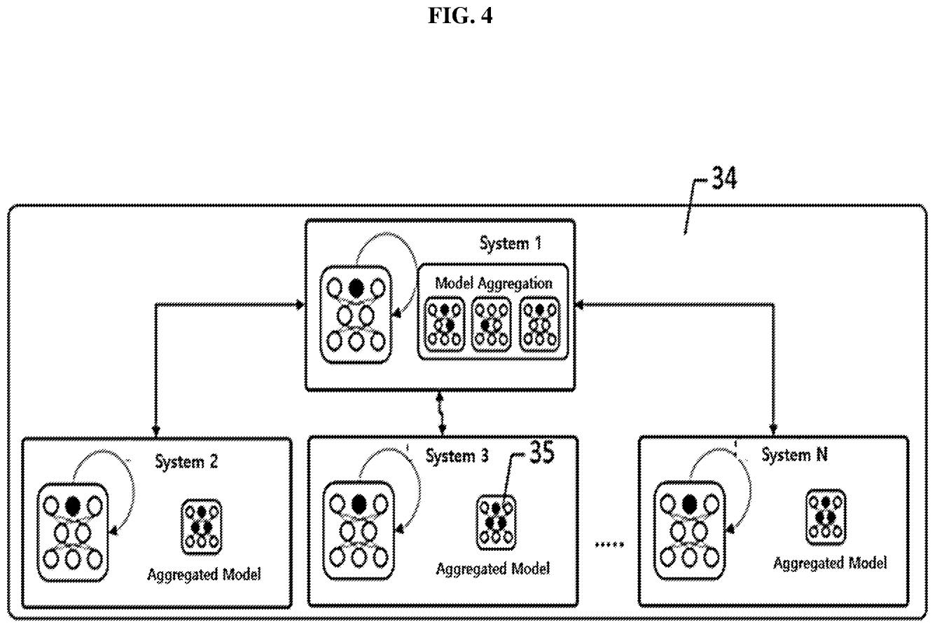

FIG. 4 illustrates a basic federated learning method related to model aggregation. In detail, FIG. 4 illustrates one of aggregation methods available for a central system (System 1) to collect model information from other systems. Here, the central system updates a model of the central system through aggregation with other models. Next, the central system distributes updated model information (aggregated model) to all of the other systems. Each system may independently update a model through aggregation with the aggregated model. Here, a aggregated model 34 may be an updated model based on a model aggregation process, such as federated averaging.

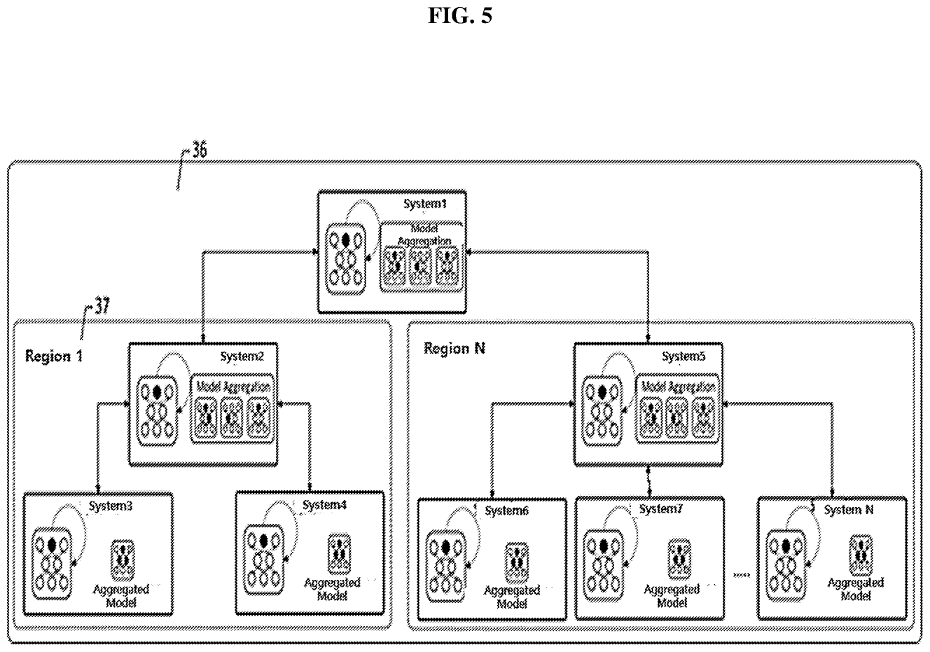

FIG. 5 illustrates a method of clustering systems into a plurality of regions among available aggregation methods. In each region, a region leader (e.g., System 2 and System 5) collects model information from other members. Next, each system may independently update a model through aggregation with other models. Here, a hierarchical federated learning model 35 may be configured as a federated learning system based on a hierarchical structure. Here, a region may be configured based on a geographical location of, for example, a city and a country. However, it is provided as an example only. The region may be dynamically configured based on a transmission route and a transmission speed of a disease to be predicted.

Meanwhile, the symptom-disease prediction system according to the present disclosure may be configured to expand input features to detect a new disease. Referring to FIGS. 1 to 5, if a new disease occurs at a time (t+1), input features need to be expanded to detect the new disease. Therefore, the present disclosure expands a disease and symptom dictionary based on findings related to the new disease. Next, the model is trained using only (t+1) data and, after training, the model may detect existing diseases with existing symptoms and new diseases with the existing symptoms and new symptoms. The same process may be repeated every time a new disease occurs.

The evolving symptom-disease prediction system and prediction method for the smart healthcare decision support system according to the present disclosure have the following technical features. Meanwhile, the present disclosure is not limited to the following primary technical features and the present disclosure may be expanded based on the primary technical features. That is, the present disclosure may be defined as the evolving symptom-disease prediction system based on increasing various information types with using a minimum amount of personal information.

a) The privacy aware computer system 1 present in each hospital updates a machine learning model without sharing patient information.

b) The symptom-disease prediction system includes two major logical components, for example, the server 4 and the client 14.

c) The client 14 that is a computer apparatus may perform an arithmetic operation.

d) The external data and communication management module 5 that is one of components of the server manages sharing of machine learning model-related information between geographically distributed systems 1 and model aggregation processes.

e) The external data and communication management module 5 that is one of components of the server may perform a task with various model aggregation processes as follows. The server may perform a task with various model aggregation processes, such as the peer to peer federated learning 30, the classic federated learning 34, and the hierarchical federal federation learning 36.

f) The data storage (Data Store) 7 that is one of components of the server 4 stores various data types of a medical device and input information of a user.

g) The model storage (Models Store) 8 that is one of components of the server 4 stores machine learning models with respect to the training and calibration module 11 and the utilization pipeline 16.

h) The internal management module 12 that is one of components of the server 4 may collect data from the client for model calibration and constructs the evolving utilization pipeline 16 to provide a service to the client.

i) The evolving training and calibration pipeline 15 constructed by the training and calibration module 11 may expand a pipeline along the input features 18, 22, and 24 that are expanding.

j) The utilization pipeline 16 constructed by the internal management module 12 may expand based on the expanding input features 18, 22, and 24.

k) The evolving utilization pipeline 16 collects information from the dashboard 27 and stores the collected information in the data storage 7 for model calibration.

l) The API 12 manages communication between the client 14 and the server 4 and provides a service, such as, for example, disease prediction.

Hereinafter, the evolving symptom-disease prediction system for the smart healthcare decision support system is described based on the aforementioned major technical features according to the present disclosure. Referring to FIGS. 1 to 5, the symptom-disease prediction system may include the client 14 and the server 4.

The client 14 may be configured to transmit data related to symptom information. Also, the server 4 may be configured to detect and predict a disease based on the data related to the symptom information.

As described above, the server 4 may be configured to include all of the components shown in FIG. 1. Depending on applications, the server 4 may include a portion of the components. The server 4 may include the data storage 7, the model storage 8, and a processor. Here, the processor may be configured to include at least one of the preprocessing module 10, the training and calibration module 11, and the internal application management module 12. Depending on applications, the processor may be configured to include at least one of the aforementioned modules and another control module.

The data storage 7 may be configured to store medical device data related to a medical device and user information including symptom information. The model storage 8 may be configured to store a machine learning model for disease prediction through interaction with the training and calibration pipeline associated with training and calibration for a user data set and the utilization pipeline associated with the disease prediction.

The processor may determine whether a disease predicted based on the machine learning model is an existing disease and a new disease. When the predicted disease is determined as the existing predicted disease and the new disease, the processor may control the machine learning model to be updated by aggregating the machine learning model with other models through a model aggregation process shared with other medical institutions.

With respect to the peer-to-peer scheme of FIG. 3, the server 4 may update the machine learning model stored in the data storage based on local collection data related to the new disease. The server 4 may finally update the machine learning model stored in the data storage based on collection data related to the new disease received from a plurality of medical institutions. The server 4 may perform detection and prediction of the new disease through the finally updated machine learning model and may distribute updated model information to a plurality of medical institution servers corresponding to the plurality of medical institutions.

With respect to the classical federated learning scheme of FIG. 4, the server 4 may update the machine learning model stored in the data storage based on local collection data related to the new disease. The server 4 may finally update the machine learning model stored in the data storage based on collection data related to the new disease received from a central system that is a representative medical institution among the plurality of medical institutions. The server 4 may perform detection and prediction of the new disease through the finally updated machine learning model and may distribute updated model information to a server of the representative medical institution.

Here, the server 4 may update the machine learning model stored in the data storage based on local collection data related to the new disease. The server 4 may finally update the machine learning model stored in the data storage based on collection data related to the new disease received from a subsystem that is a representative medical institution of a group to which the server 4 belongs. The server 4 may perform detection and prediction of the new disease through the finally updated machine learning model and may distribute updated model information to a server of the subsystem that is the representative medical institution of the group.

With respect to the hierarchical federated learning scheme of FIG. 5, the server 4 may evaluate whether to update the machine learning model based on the collection data related to the new disease received from the subsystem. When update is evaluated to not be performed, the server 4 may receive second collection data from the central system interacting with subsystems of each group through the subsystem. The server 4 may re-update the machine learning model stored in the data storage based on the second collection data. The server 4 may perform detection and prediction of the new disease through the re-updated machine learning model and may distribute re-updated model information to the server of the subsystem that is the representative medical institution of the group.

Referring to FIGS. 1 to 5, the server 4 may detect a first point in time at which the predicted disease is determined as the existing predicted disease and the new disease. The server 4 may control the machine learning model to be trained based on data acquired after the first point in time, at a second point in time after the first point in time.

Meanwhile, the server 4 may be configured to not use sensitive user information of other medical institutions. Here, the server 4 may detect the first point in time at which the predicted disease is determined as the existing predicted disease and the new disease. The server 4 may control the machine learning model to be trained based on user data of a corresponding medical institution when performing training and calibration of the user data set, at the second point in time after the first point in time. The server 4 may control the machine learning model to be trained based on disease data of a corresponding medical institution and other medical institutions acquired after the first point in time, at the second point in time.

Meanwhile, the server 4 may be configured to provide disease detection results and a variety of information to the client 14. The server 4 may perform prediction and detection of the new disease using the updated machine learning model. The server 4 may transmit, to the client that is a user terminal associated with a user of which the new disease is detected, detection results about the new disease and diagnostic results and prevention information according to body and health information of the user.

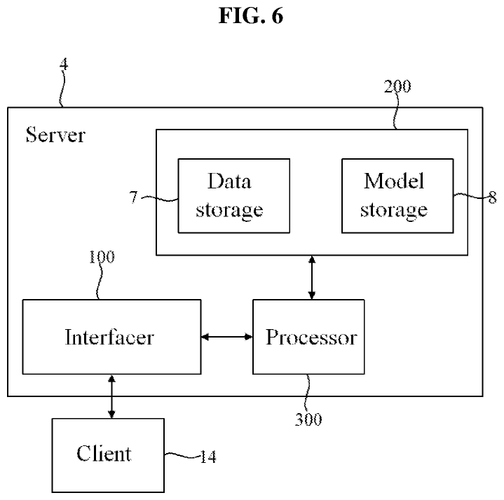

Hereinafter, a server of an evolving symptom-disease prediction system for a smart healthcare decision support system according to another aspect of the present disclosure is described. Here, FIG. 6 is a configuration diagram illustrating interaction between a server of an evolving symptom-disease prediction system for a smart healthcare decision support system and a client according to the present disclosure. Referring to FIGS. 1 to 6, the server 4 may be configured to include an interface 100, a storage 200, and a processor 300. Here, the interface 100 may be configured to transmit data related to symptom information 14 and receive disease detection results and diagnosis results from the client.

The storage 200 may be configured to store medical device data related to a medical device and user information including symptom information, and to store a machine learning model for disease prediction through interaction with the training and calibration pipeline associated with training and calibration for a user data set and the utilization pipeline associated with the disease prediction. Meanwhile, when a disease predicted based on the machine learning model is determined as an existing predicted disease and a new disease, the processor 300 may control the machine learning model to be updated by aggregating the machine learning model with other models through a model aggregation process shared with other medical institutions.

The storage 200 may be configured to include the data storage 7 and the model storage 8. The data storage 7 may be configured to store the medical device data related to the medical device and the user information including the symptom information. The model storage 8 may be configured to store the machine learning model for the disease prediction through interaction with the training and calibration pipeline associated with training and calibration for the user data set and the utilization pipeline associated with the disease prediction.

The processor 300 may update the machine learning model stored in the data storage 7 based on local collection data related to the new disease. The processor 300 may finally update the machine learning model stored in the data storage based on the collection data related to the new disease received from the plurality of medical institutions. The processor 300 may perform detection and prediction of the new disease through the finally updated machine learning model and may distribute the updated model information to a plurality of medical institution servers corresponding to the plurality of medical institutions.

With respect to the classical federated learning, the processor 300 may update the machine learning model stored in the data storage based on local collection data related to the new disease. The processor 300 may finally update the machine learning model stored in the data storage based on collection data related to the new disease received from a central system that is a representative medical institution among the plurality of medical institutions. The processor 300 may perform detection and prediction of the new disease through the finally updated machine learning model and distribute updated model information to a server of the representative medical institution.

With respect to the hierarchical federated learning, the processor 300 may update the machine learning model stored in the data storage 7 based on local collection data related to the new disease. The processor 300 may finally update the machine learning model stored in the data storage based on collection data related to the new disease received from a subsystem that is a representative medical institution of a group to which the server 4 belongs. The processor 300 may perform detection and prediction of the new disease through the finally updated machine learning model and may distribute updated model information to a server of the subsystem that is the representative medical institution of the group.

With respect to the hierarchical federated learning, when update of the machine learning model is evaluated to not be performed based on the collection data related to the new disease received from the subsystem, the processor 300 may receive second collection data from the central system interacting with subsystems of each group through the subsystem. The processor 300 may re-update the machine learning model stored in the data storage based on the second collection data. The processor 300 may perform detection and prediction of the new disease through the re-updated machine learning model and distribute re-updated model information to the server of the subsystem that is the representative medical institution of the group.

With respect to the aforementioned various learning methods, the processor 300 may detect a first point in time at which the predicted disease is determined as the existing predicted disease and the new disease. The processor 300 may control the machine learning model to be trained based on data acquired after the first point in time, at a second point in time after the first point in time.

As described above, the processor 300 may detect the first point in time at which the predicted disease is determined as the existing predicted disease and the new disease. The processor 300 may control the machine learning model to be trained based on user data of a corresponding medical institution when performing training and calibration of the user data set, at the second point in time after the first point in time. The processor 300 may control the machine learning model to be trained based on disease data of a corresponding medical institution and other medical institutions acquired after the first point in time, at the second point in time.

The processor 300 may perform prediction and detection of the new disease using the updated machine learning model. The processor 300 may transmit, to the client that is a user terminal associated with a user of which the new disease is detected, detection results about the new disease and diagnostic results and prevention information according to body and health information of the user.

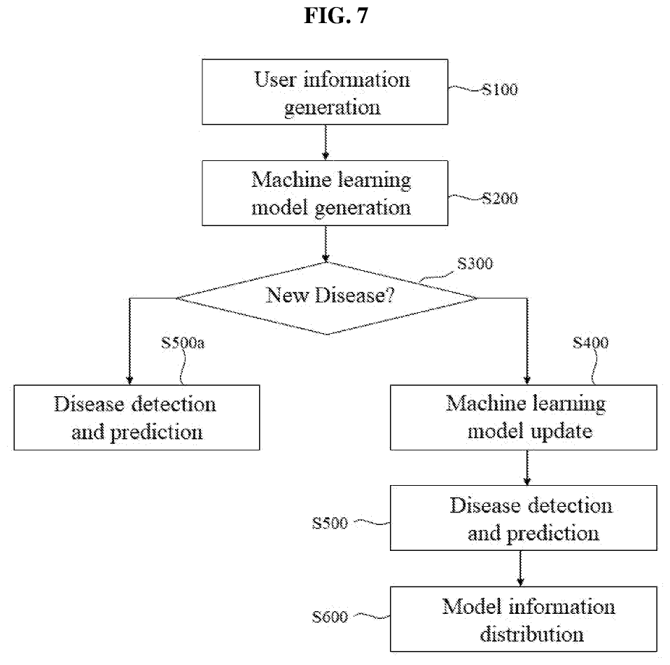

Hereinafter, an evolving symptom-disease prediction method for a smart healthcare decision support system according to still another aspect of the present disclosure is described. Here, FIG. 7 is a flowchart illustrating an evolving symptom-disease prediction method for a smart healthcare decision support system according to another aspect of the present disclosure. Here, the aforementioned description related to the symptom-disease prediction system and the server may apply to the symptom-disease prediction method through combination.

Referring to FIG. 7, the symptom-disease prediction method may include user information generation process S100, machine learning model generation process S200, disease decision process S300, and machine learning model update process S400.

In user information generation process S100, medical device data related to a medical device and user information including symptom information may be generated. In machine learning model generation process S200, a machine learning model for disease prediction may be generated through interaction with a training and calibration pipeline associated with training and calibration for a user data set and a utilization pipeline associated with the disease prediction. In disease decision process S300, whether a disease predicted based on the machine learning model is an existing predicted disease and a new disease may be determined.

When the predicted disease is determined as the existing predicted disease and the new disease in disease decision process S300, machine learning model update process S400 may be performed. On the contrary, when the disease predicted is determined as the existing predicted disease in disease decision process S300, disease detection and prediction process S500a may be performed.

In machine learning model update process S400, the machine learning model may be controlled to be updated by aggregating the machine learning model with other models through a model aggregation process shared with other medical institutions.

Meanwhile, machine learning model update process S400 may include first update process S410 and second update process S420. In first update process S410, the machine learning model stored in the data storage may be updated based on local collection data related to the new disease. In second update process S420, the machine learning model stored in the data storage may be updated based on collection data related to the new disease received from a plurality of medical institutions.

The symptom-disease prediction method according to the present disclosure may further include disease detection and prediction process S500 and model information distribution process S600. In disease detection and prediction process S500, detection and prediction of the new disease may be performed through a finally updated machine learning model. In model information distribution process S600, the finally updated model information may be distributed to a plurality of medical institution servers corresponding to the plurality of medical institutions.

The evolving symptom-disease prediction system for the smart healthcare decision support system according to the present disclosure is described above. The technical effects of the evolving symptom-disease prediction system for the smart healthcare decision support system according to the present disclosure follow as:

According to the present disclosure, an evolving symptom-disease prediction system for a smart healthcare decision support system may provide various model aggregation methods.

According to the present disclosure, an evolving symptom-disease prediction result for a smart healthcare decision support system may enhance quality of a medical service by providing accurate symptom-disease information to a medical profession.

According to the present disclosure, it is possible to construct a mobile/web application that is a part of a smart healthcare system as a model of a symptom-disease prediction system.

The features and effects of the present disclosure will be apparent through the following detailed description described with reference to the accompanying drawings and thus, those skilled in the art to which the disclosure pertains may easily perform the technical spirit of the disclosure.

The present disclosure may make various alterations and modifications and have some example embodiments and thus, specific example embodiments are illustrated as examples and are described in the detailed description. However, the example embodiments are not construed as being limited to the disclosure and should be understood to include all changes, equivalents, and replacements within the spirit and technical scope of the disclosure.

According to software implementation, design and parameter optimization about each of components as well as procedures and functions described herein may be implemented as a separate software module. A software code may be implemented as a software application written in an appropriate program language. The software code may be stored in a memory and executed by a controller or a processor.

What is claimed is:

1. An evolving symptom-disease prediction system for a smart healthcare decision support system, the symptom-disease prediction system comprising:
a client configured to transmit data related to symptom information; and
a server configured to detect and predict a disease based on the data related to the symptom information,
wherein the server comprises:
a data storage configured to store medical device data related to a medical device and user information including symptom information;
a model storage configured to store a machine learning model for disease prediction through interaction with a training and calibration pipeline associated with training and calibration for a user data set and a utilization pipeline associated with the disease prediction; and
a processor configured to, when a disease predicted based on the machine learning model is determined as an existing predicted disease and a new disease, control the machine learning model to be updated by aggregating the machine learning model with other models through a model aggregation process shared with other medical institutions.

2. The symptom-disease prediction system of claim 1, wherein the server is configured to
update the machine learning model stored in the data storage based on local collection data related to the new disease,
finally update the machine learning model stored in the data storage based on collection data related to the new disease received from a plurality of medical institutions, and
perform detection and prediction of the new disease through the finally updated machine learning model and distribute updated model information to a plurality of medical institution servers corresponding to the plurality of medical institutions.

3. The symptom-disease prediction system of claim 2, wherein the server is configured to
update the machine learning model stored in the data storage based on local collection data related to the new disease,
finally update the machine learning model stored in the data storage based on collection data related to the new disease received from a central system that is a representative medical institution among the plurality of medical institutions, and
perform detection and prediction of the new disease through the finally updated machine learning model and distribute updated model information to a server of the representative medical institution.

4. The symptom-disease prediction system of claim 3, wherein the server is configured to
update the machine learning model stored in the data storage based on local collection data related to the new disease,
finally update the machine learning model stored in the data storage based on collection data related to the new disease received from a subsystem that is a representative medical institution of a group to which the server belongs, and
perform detection and prediction of the new disease through the finally updated machine learning model and distribute updated model information to a server of the subsystem that is the representative medical institution of the group.

5. The symptom-disease prediction system of claim 4, wherein the server is configured to
when update of the machine learning model is evaluated to not be performed based on the collection data related to the new disease received from the subsystem, receive second collection data from the central system interacting with subsystems of each group through the subsystem,
re-update the machine learning model stored in the data storage based on the second collection data, and
perform detection and prediction of the new disease through the re-updated machine learning model and distribute re-updated model information to the server of the subsystem that is the representative medical institution of the group.

6. The symptom-disease prediction system of claim 2, wherein the server is configured to
detect a first point in time at which the predicted disease is determined as the existing predicted disease and the new disease, and
control the machine learning model to be trained based on data acquired after the first point in time, at a second point in time after the first point in time.

7. The symptom-disease prediction system of claim 2, wherein the server is configured to
detect a first point in time at which the predicted disease is determined as the existing predicted disease and the new disease, and
control the machine learning model to be trained based on user data of a corresponding medical institution when performing training and calibration of the user data set, at a second point in time after the first point in time.

8. The symptom-disease prediction system of claim 7, wherein the server is configured to control the machine learning model to be trained based on disease data of a corresponding medical institution and other medical institutions acquired after the first point in time, at the second point in time.

9. The symptom-disease prediction system of claim 2, wherein the server is configured to
perform prediction and detection of the new disease using the updated machine learning model, and
transmit, to the client that is a user terminal associated with a user of which the new disease is detected, detection results about the new disease and diagnostic results and prevention information according to body and health information of the user.

10. A server of an evolving symptom-disease prediction system for a smart healthcare decision support system, the server comprising:
a storage configured to store medical device data related to a medical device and user information including symptom information, and to store a machine learning model for disease prediction through interaction with a training and calibration pipeline associated with training and calibration for a user data set and a utilization pipeline associated with the disease prediction; and
a processor configured to, when a disease predicted based on the machine learning model is determined as an existing predicted disease and a new disease, control the machine learning model to be updated by aggregating the machine learning model with other models through a model aggregation process shared with other medical institutions.

11. The server of claim 10, wherein the storage comprises:
a data storage configured to store the medical device data related to the medical device and the user information including the symptom information; and
a model storage configured to store the machine learning model for the disease prediction through interaction with the training and calibration pipeline associated with training and calibration for the user data set and the utilization pipeline associated with the disease prediction.

12. The server of claim 11, wherein the processor is configured to
update the machine learning model stored in the data storage based on local collection data related to the new disease,
finally update the machine learning model stored in the data storage based on collection data related to the new disease received from a plurality of medical institutions, and
perform detection and prediction of the new disease through the finally updated machine learning model and distribute the updated model information to a plurality of medical institution servers corresponding to the plurality of medical institutions.

13. The server of claim 12, wherein the processor is configured to
update the machine learning model stored in the data storage based on local collection data related to the new disease,
finally update the machine learning model stored in the data storage based on collection data related to the new disease received from a central system that is a representative medical institution among the plurality of medical institutions, and
perform detection and prediction of the new disease through the finally updated machine learning model and distribute updated model information to a server of the representative medical institution.

14. The server of claim 13, wherein the processor is configured to
update the machine learning model stored in the data storage based on local collection data related to the new disease,
finally update the machine learning model stored in the data storage based on collection data related to the new disease received from a subsystem that is a representative medical institution of a group to which the server belongs, and
perform detection and prediction of the new disease through the finally updated machine learning model and distribute updated model information to a server of the subsystem that is the representative medical institution of the group.

15. The server of claim 14, wherein the processor is configured to
when update of the machine learning model is evaluated to not be performed based on the collection data related to the new disease received from the subsystem, receive second collection data from the central system interacting with subsystems of each group through the subsystem,
re-update the machine learning model stored in the data storage based on the second collection data, and
perform detection and prediction of the new disease through the re-updated machine learning model and distribute re-updated model information to the server of the subsystem that is the representative medical institution of the group.

16. The server of claim 12, wherein the processor is configured to
detect a first point in time at which the predicted disease is determined as the existing predicted disease and the new disease, and
control the machine learning model to be trained based on data acquired after the first point in time, at a second point in time after the first point in time.

17. The server of claim 12, wherein the processor is configured to
detect a first point in time at which the predicted disease is determined as the existing predicted disease and the new disease,
control the machine learning model to be trained based on user data of a corresponding medical institution when performing training and calibration of the user data set, at a second point in time after the first point in time, and
control the machine learning model to be trained based on disease data of a corresponding medical institution and other medical institutions acquired after the first point in time, at the second point in time.

18. The server of claim 12, wherein the processor is configured to
perform prediction and detection of the new disease using the updated machine learning model, and
transmit, to a client that is a user terminal associated with a user of which the new disease is detected, detection results about the new disease and diagnostic results and prevention information according to body and health information of the user.

19. An evolving symptom-disease prediction method for a smart healthcare decision support system, the symptom-disease prediction method comprising:
a user information generation process of generating medical device data related to a medical device and user information including symptom information;
a machine learning model generation process of generating a machine learning model for disease prediction through interaction with a training and calibration pipeline associated with training and calibration for a user data set and a utilization pipeline associated with the disease prediction;
a disease decision process of determining whether a disease predicted based on the machine learning model is an existing predicted disease and a new disease; and
a machine learning model update process of, when the predicted disease is determined as the existing predicted disease and the new disease, controlling the machine learning model to be updated by aggregating the machine learning model with other models through a model aggregation process shared with other medical institutions.

20. The symptom-disease prediction method of claim 19, wherein the machine learning model update process comprises:
a first update process of updating a machine learning model stored in a data storage based on local collection data related to the new disease; and
a second update process of updating the machine learning model stored in the data storage based on collection data related to the new disease received from a plurality of medical institutions, and
the method further comprises:
a disease detection and prediction process of performing detection and prediction of the new disease through a finally updated machine learning model; and
a model information distribution process of distributing the finally updated model information to a plurality of medical institution servers corresponding to the plurality of medical institutions.
